(19) United States
(12) Patent Application Publication
(10) Pub. No.: US 2003/0124124 A1
(43) Pub. Date: Jul. 3, 2003

(54) Therapeutic modulation of chemokines using a helminth parasite chemokine-binding protein

(76) Inventors: Padraic Fallon, Dublin 2 (IE); Philip Smith, Dublin 2 (IE); Antonio Alcami, Cambridge (GB)

Correspondence Address: NIXON & VANDERHYE, PC, 1100 N GLEBE ROAD, 8TH FLOOR, ARLINGTON, VA, 22201-4714, US

(21) Appl. No.: 10/293,950

(22) Filed: Nov. 14, 2002

Related U.S. Application Data
(63) Continuation-in-part of application No. PCT/GB02/02963, filed on Jun. 27, 2002.

(30) Foreign Application Priority Data
Jun. 27, 2001 (GB) 0115780.9

(51) Int. Cl. A61K 39/395; C07H 21/04; C12P 21/02; C12N 5/06; C07K 16/24

(52) U.S. Cl. 424/145.1; 435/69.1; 435/320.1; 530/388.23; 435/335; 536/23.53

(57) ABSTRACT

ES-2 polypeptide encoding nucleic acid has been cloned from Schistosoma mansoni and shown to bind and inhibit chemokine function, including CC chemokines such as MIP-1α and/or RANTES, CXC chemokines such as IL-8, C chemokines and CX₃C chemokines, both in vivo and in vitro. The polypeptide is useful in therapy of inflammatory disorders and other diseases, and in assaying for substances with ability to potentiate or inhibit its anti-chemokine function, which substances have additional therapeutic potential.

The present invention concerns molecules useful in modulation of chemokine-mediated diseases. It relates to helminth-derived chemokine binding molecules, especially a molecule obtainable from the trematode parasite Schistosoma mansoni that binds chemokines and impairs chemokine function. It relates to cloning and use of encoding nucleic acid encoding the ES-2 polypeptide of Schistosoma mansoni, and the ability of the ES-2 molecule to bind and inhibit chemokine function, both in vitro and in vivo as demonstrated experimentally.

A dominant factor in the evolution of the immune system is the control of infectious diseases (bacteria, virus and parasites). The co-evolution of humans and pathogens has resulted in adaptation of the immune system to tolerate potentially lethal infections. Pathogens have evolved various strategies to modulate the functions of the host's immune system. In all major human pathogens immunomodulatory genes have been selected, with the products of such genes manipulating various aspects of the immune system, e.g. for immune evasion by viruses see (1). Thus immunomodulatory molecules (IMs) produced by these genes can modify the immune system.

As a group, helminth parasite infections have exerted marked selective pressure on the immune system. The co-evolution of humans and helminth parasites has resulted in adaptation of the immune system to tolerate chronic helminth infections, often for decades, without overt morbidity. Schistosome spp. are human trematode parasites which infect over 200 million people, and are a potent modulator of the immune system. Schistosome infection of man has been shown to modulate responses to vaccination and may reduce the incidence of atopy. Injection of mice with eggs from Schistosoma mansoni renders the mice refractory to viral challenge, diabetes and nematode parasite challenge. Antigens synthesized by schistosome eggs and secreted into host tissue are bioactive IMs. The schistosome egg is a potent inducer of cellular inflammation characterized by tissue eosinophilia, IgE synthesis, secretion of type 2 cytokines (Interleukin [IL]-4, IL-5, IL-9, IL-10 and IL-13) and chemokines (2). In mice the inflammation elicited by the schistosome egg is associated with temporal tissue expression of protein and mRNAs from various chemokines (3).

Chemokines are small proteins that are secreted into the body and mediate diverse immunological responses including influencing cellular migration, positioning and degranulation, angiogenesis and Th1/Th2 cytokine responses. Chemokines are divided into four families based on the number and configuration of conserved cysteine residues near the N-terminus into: CC chemokines [including macrophage inflammatory protein (MIP)-1α, RANTES (regulated on activation, normal T-cell expressed and secreted) and monocyte chemotactic protein (MCP)-1], CXC chemokines [IL-8 and growth-related oncogene (GRO)-α], the C chemokine lymphotactin and the CX₃C chemokine fractalkine. As chemokines are central to the elicitation of inflammation and also the control of various infectious agents they are considered good therapeutic targets for the control of various human diseases (4).

The present inventors have identified a novel molecule (ES-2) that is secreted from S. mansoni eggs that can bind members of at least two families of chemokines (CC and CXC). ES-2 was also shown in the experiments described herein both to block the interaction of chemokines with cellular receptors and to reduce inflammation in vivo. The ES-2 molecule corresponds with a molecule previously shown to be secreted from schistosome eggs. Based on molecular weight, in native and reduced SDS-PAGE, ES-2 is a previously described egg glycoprotein that is present in the eggs of the three major of species of schistosome that infects humans (6, 7). Previous interest in ES-2 related to its use as a diagnostic antigen as the native molecule is produced by various Schistosoma species and is recognized by antibodies in sera from humans infected with schistosomiasis. Research into ES-2 as a potential diagnostic antigen declined due to the availability of more sensitive and commercial available diagnostic techniques. The present invention is based on the novel finding of function for ES-2 as a chemokine-binding molecule.

This invention relates to the use of ES-2 polypeptides or the active chemokine-binding domain(s) thereof in treatment of diseases and disorders where chemokines or analogues are implicated. Diseases mediated by chemokines include pulmonary inflammation (e.g. asthma and rhinitis), skin inflammation (e.g. psoriasis), atherosclerosis, multiple sclerosis, arthritis, neoplasia, sepsis, bacterial and viral infections (including mycobacteria and HIV) and colitis (for review see (5)). Administration of ES-2 polypeptides is useful to neutralize circulating chemokines and prevent binding to chemokine receptors and thereby ameliorate chemokine-mediated responses. Similarly, nucleic acid encoding ES-2 polypeptides may be inserted into vector systems for controlled cell or tissue expression in an organism. Chemokines that are inhibited by ES-2 that are associated with disease conditions include MIP-1α, involved for example in allergies and experimental autoimmune encephalomyelitis; IL-8, involved for example in psoriasis and atherosclerosis, and RANTES, involved for example in allergies and glomerulonephritis.

### BRIEF DESCRIPTION OF THE FIGURES

FIG. 1 shows that ES-2 impedes the specific binding of MIP-1α and IL-8 to U937 cells. Various concentrations of ES-2 (2500-7000 ng/ml) were incubated with ¹²⁵I-MIP-1α (FIG. 1A) or ¹²⁵I-IL-8 (FIG. 1B) prior to addition to U937 cells. OVA was added as a control glycoprotein. ¹²⁵I-chemokine bound to U937 cells were measured. Data are presented as the percentage of ¹²⁵I-chemokine bound chemokine relative to the values in cells incubated with ¹²⁵I-chemokine with no competitor. Closed circles are for ES-2, open circles for OVA. Values are mean from duplicate samples.

FIG. 2 shows that ES-2 blocks activation of PBMCs by RANTES. PBMCs that had been pre-labeled with Fluro-4AM were activated by RANTES-alone, or RANTES that had been pre-incubated with ES-2 or OVA (250-2000 ng/ml). The activation of cells was detected by Ca⁺⁺ flux using a flow cytometer. Data are presented as the % inhibition of activation by chemokine+competitors relative to activation in RANTES-alone stimulated cells. Dark shaded bars are for ES-2, open bars for OVA.

FIG. 3 shows that ES-2 blocks IL-8 mediated migration of human neutrophils. IL-8 was pre-incubated with different concentrations of ES-2 or OVA (250-5000 ng/ml) prior to addition to transwell migration plate. Control groups included the addition of no IL-8 (Blank) and IL-8 alone. Numbers of migrated neutrophils are expressed as mean and SE from triplicate wells. Dark shaded bars are for ES-2, open bars for OVA and intermediate grey shaded controls.

FIG. 4 shows that ES-2 blocks in vivo contact hypersensitivity (CHS). Mice were sensitized by the application of 2,4-dintrofluorobenzenes (DNFB) to the shaved abdomen. Mice were injected i.v. with PBS, ES-2 or OVA (15 μg) 5 days later. CHS was elicited 20 minutes after injections by the application of DNFB solution to the right ear and vehicle to the left ear. The increased inflammation was monitored 24 hours later. Data are from 6-7 mice per group and are presented as mean and SE. Student's t-test was used to determine statistical differences between groups.

FIG. 5 shows results of experiments demonstrating inhibition of IL-8 mediated neutrophil infiltration of murine air pouches by intravenous (iv) injection of recombinant ES-2 (rES-2). Neutrophil infiltration was measured following injection of air pouches with PBS or IL-8. Mice were injected iv with rES-2 or ovalbumin. Data are mean+SD from 6 mice per group.

FIG. 6 shows results experiments using flow cytometry detection of GR-1 positive neutrophils (expressed as a percentage) in air pouch lavage of mice. FIG. 6A shows results for air pouch treatment with PBS. FIG. 6B shows results for air pouch treatment with IL-8 and OVA. FIG. 6C shows results for air pouch treatment with IL-8 and rES-2.

SEQ ID NO. 1 provides the nucleotide sequence and SEQ ID NO. 2 the deduced amino acid sequence of ES-2. The translated amino acid sequence is shown above the nucleotide sequence. Amino acids are numbered on the right.

The present invention is based on identification of ES-2 as having ability to bind CC chemokines such as MIP-1α and/or RANTES and CXC chemokines such as IL-8, and ability to inhibit chemokine activity. Furthermore, it is based on the demonstration that ES-2 is useful in vivo for inhibition of chemokine activity in disorders mediated by chemokines. This provides for clinical uses of ES-2. Furthermore, in seeking treatments for schistosome diseases, the interaction between ES-2 and chemokines allows for screens and assays for molecules that modulate the interaction, either potentiating or inhibiting the binding.

In accordance with various aspects and embodiments of the present invention, ES-2 polypeptide, preferably in an isolated or purified form, may be used in a variety of contexts.

One aspect of the present invention provides a method of inhibiting activity of a chemokine, the method comprising bringing the chemokine into contact with an ES-2 polypeptide of the invention. Such a method may comprise administering the ES-2 polypeptide to an individual, or it may take place in vitro, for instance in cell culture. ES-2 polypeptide may be provided to cells in vitro or in vivo by means of encoding nucleic acid, wherein the coding sequence is under control of appropriate regulatory sequences for production of the encoded ES-2 polypeptide by expression from the nucleic acid.

ES-2 polypeptide may bind and preferably inhibit a chemokine. Chemokines bound and inhibited by ES-2 in accordance with the present invention include CC chemokines such as MIP-1α and/or RANTES, CXC chemokines such as IL-8, C chemokines and CX₃C chemokines.

Chemokines are implicated in a number of diseases and disorders of the inflammatory system, including those mentioned above.

In accordance with a further aspect of the present invention, there is provided a method of treating a disease or disorder involving chemokine activity, the method comprising administering an ES-2 polypeptide to an individual with such a disease or disorder. The disease or disorder may involve activity of a CC chemokine such as MIP-1α and/or RANTES, and/or a CXC chemokine such as IL-8.

Diseases and disorders that may be treated in accordance with the present invention include allergic diseases, for example asthma, rhinitis and anaphylaxis, chronic pulmonary diseases, psoriasis, atherosclerosis, multiple sclerosis, arthritis, neoplasia, sepsis, infectious diseases including for example Schistosomiasis, malaria, tuberculosis and HIV, inflammatory bowel disease, diabetes, colitis and transplant rejection.

In a further aspect, the present invention provides ES-2, or a composition comprising ES-2 polypeptide (e.g. also comprising a pharmaceutically acceptable vehicle, diluent excipient or carrier) for use in a method of treatment of the human or animal body by therapy. The ES-2 polypeptide or composition comprising the ES-2 polypeptide may be for use in a method of treatment of any disease or disorder involving chemokine activity, e.g. as set out in the preceding paragraph. The disease or disorder may involve activity of a CC chemokine such as MIP-1α and/or RANTES, and/or a CXC chemokine such as IL-8.

In a further aspect, the present invention provides for use of an ES-2 polypeptide in the manufacture of a medicament for treating a disease or disorder involving chemokine activity.

A further aspect of the present invention provides a method of making a pharmaceutical composition comprising admixing a ES-2 polypeptide with a pharmaceutically acceptable excipient, vehicle, diluent or carrier, and optionally other ingredients.

Furthermore, the recombinantly produced polypeptide may be used in a diagnostic product, useful for instance in detection of antibodies in sera taken from individuals who have, may have or are suspected of having a schistosome infection. The polypeptide may be used in any assay format available in the art for detection of antibodies in a sample, or determination of the level of antibodies in a sample, allowing for use of the information in diagnosis or prognosis and/or in monitoring progress of disease or of treatment of disease.

As discussed further below, the present invention also encompasses substances that are able to affect the ability of an ES-2 polypeptide to bind and/or inhibit activity of a CC chemokine such as MIP-1α and/or RANTES, and/or a CXC chemokine such as IL-8, for instance potentiate binding and/or inhibition or inhibit such binding and/or inhibition. Such substances may be useful in treatment of disease, especially schistosome disease. A substance that potentiates the effect of an ES-2 polypeptide may be used to potentiate the effect of ES-2 polypeptide in therapy (for example in therapy of an inflammatory disorder), e.g. by combined, simultaneous or sequential administration.

In various further aspects, the present invention thus provides a pharmaceutical composition, medicament, drug or other composition for such a purpose, the composition comprising a ES-2 polypeptide and/or a substance able to affect the ability of an ES-2 polypeptide to bind and/or inhibit a chemokine, the use of such a polypeptide and/or substance in a method of medical treatment, a method comprising administration of such a polypeptide and/or substance to a patient, e.g. for treatment (which may include preventative treatment) of a medical condition, e.g. a condition associated with one or more chemokine activities, use of such a polypeptide and/or substance in the manufacture of a composition, medicament or drug for administration for such a purpose, and a method of making a pharmaceutical composition comprising admixing such a polypeptide and/or substance with a pharmaceutically acceptable excipient, vehicle, diluent or carrier, and optionally other ingredients.

The polypeptide and/or substance may be used as sole active agents or in combination with one another or with any other active substance.

Whatever the polypeptide and/or substance used in a method of medical treatment of the present invention, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A polypeptide, substance or composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Administration may be by aerosol for pulmonary delivery.

Administration may be by topical application to the skin.

Examples of techniques and protocols mentioned above can be found in Remington's Pharmaceutical Sciences, 16th edition, Osol, A. (ed), 1980.

The substance or composition may be administered in a localised manner to a desired site or may be delivered in a manner in which it targets cells.

Targeting therapies may be used to deliver the active substance more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Instead of administering such substances directly, they may be produced in the target cells by expression from an encoding nucleic acid introduced into the cells, e.g. from a viral vector. The vector may be targeted to the specific cells to be treated, or it may contain regulatory elements which are switched on more or less selectively by the target cells.

Nucleic acid encoding the active ingredient may thus be used in methods of gene therapy, for instance in treatment of individuals, e.g. with the aim of preventing or curing (wholly or partially) a disorder.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired peptide. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors in gene therapy other known methods of introducing nucleic acid into cells includes mechanical techniques such as microinjection, transfer mediated by liposomes and receptor-mediated DNA transfer.

Isolated or purified ES-2 polypeptide is generally used in pharmaceutical contexts.

The present invention provides for production and use of pure ES-2 polypeptide. A preferred polypeptide of the invention comprises or consists of the amino acid sequence of SEQ ID NO. 2, and is provided as a further aspect of the present invention.

The amino acid sequence of SEQ ID NO. 2 is encoded by the nucleotide sequence of SEQ ID NO. 1, and nucleic acid encoding a ES-2 polypeptide is provided as an aspect of the invention. Polypeptides of the invention and encoded by nucleic acid of the invention include those encoded by alleles of the sequence, and homologues of other Schistosome species that produce native ES-2, including S. japonicum, S. haemotobium, S. bovis, as well as fragments of such polypeptides as discussed further below. The primary sequence of the ES-2 protein will be substantially similar to that of SEQ ID NO. 2 and may be determined by routine techniques available to those of skill in the art. In essence, such techniques include using polynucleotides derived from SEQ ID NO. 1 as probes to recover and to determine the sequence of the ES-2 gene in other species. A wide variety of techniques are available for this, for example PCR amplification and cloning of the gene using a suitable source of mRNA, or by methods including obtaining a cDNA library from the schistosome, probing said library with a polynucleotide of the invention under stringent conditions, and recovering a cDNA encoding all or part of the ES-2 polypeptide of that schistosome. Where a partial cDNA is obtained, the full length coding sequence may be determined by primer extension techniques.

An “active portion” of the polypeptides means a peptide which is less than said full length polypeptide, but which retains its essential biological activity. In particular, the active portion retains the ability to bind to and preferably inhibit activity of a chemokine, e.g. a CC chemokine such as MIP-1α and/or RANTES, and/or a CXC chemokine such as IL-8. Fragments consisting of or comprising active portions of ES-2 polypeptides are useful as ES-2 polypeptides in accordance with the present invention.

The present invention includes a polypeptide including a portion of an ES-2 polypeptide, which polypeptide may include heterologous amino acids, such as an identifiable sequence or domain of another protein, or a histidine tag or other tag sequence, and the invention includes a polypeptide consisting essentially of a portion of a ES-2 polypeptide able to bind and preferably inhibit a chemokine.

Isolated ES-2 polypeptides of the invention will be those as defined herein in isolated form, free or substantially free of material with which it is naturally associated such as other polypeptides with which it is found in the cell. The polypeptides may of course be formulated with diluents or excipients and still for practical purposes be isolated—for example the polypeptides may be mixed with a carrier if used to coat microtitre plates for use in immunoassays, and may be mixed with a pharmaceutically acceptable vehicle, excipient, diluent or carrier when employed in a method of treatment as discussed. The polypeptides may be glycosylated, either naturally or by systems of heterologous eukaryotic cells, or they may be (for example if produced by expression in a prokaryotic cell) unglycosylated. The term “lacking native glycosylation” may be used with reference to a polypeptide which either has no glycosylation (e.g. following production in a prokaryotic cell) or has a pattern of glycosylation that is not the native pattern, e.g. as conferred by expression in a particular host cell type (which may be CHO cells).

Polypeptides of the invention may be modified for example by the addition of a signal sequence to promote their secretion from a cell or of histidine residues to assist their purification. Fusion proteins may be generated that incorporate (e.g.) six histidine residues at either the N-terminus or C-terminus of the recombinant protein. Such a histidine tag may be used for purification of the protein by using commercially available columns which contain a metal ion, either nickel or cobalt (Clontech, Palo Alto, Calif., USA). These tags also serve for detecting the protein using commercially available monoclonal antibodies directed against the six histidine residues (Clontech, Palo Alto, Calif., USA).

Polypeptides which are amino acid sequence variants, alleles, derivatives or mutants are also provided by the present invention, such forms having at least 35% sequence identity, for example at least 50%, 60%, 70%, 80%, 90%, 95%, 98% or 99% sequence identity to SEQ ID NO. 2. A ES-2 polypeptide which is a variant, allele, derivative or mutant may have an amino acid sequence which differs from that given in SEQ ID NO. 2 by one or more of addition, substitution, deletion and insertion of one or more (such as from 1 to 20, for example 2, 3, 4, or 5 to 10) amino acids.

A polypeptide according to the present invention may be isolated and/or purified (e.g. using an antibody) for instance after production by expression from encoding nucleic acid. Encoding nucleic acid is provided by the invention, as are methods for production of the encoded polypeptide by expression from the encoding nucleic acid. Polypeptides according to the present invention may also be generated wholly or partly by chemical synthesis, for example in a step-wise manner. The isolated and/or purified polypeptide may be used in formulation of a composition, which may include at least one additional component, such as a diluent.

A polypeptide according to the present invention or produced in accordance with the present invention may be used in screening for molecules which affect or modulate its ability to bind a chemokine and/or inhibit chemokine activity or function. Such molecules may be useful in a therapeutic (which may include prophylactic) context. This is discussed in detail below.

A polypeptide of the invention may be labelled with a revealing label. The revealing label may be any suitable label which allows the polypeptide to be detected. Suitable labels include radioisotopes, e.g. ¹²⁵I, enzymes, antibodies, polynucleotides and linkers such as biotin.

As noted, a preferred way of producing a polypeptide of the invention is to employ encoding nucleic acid in a suitable expression system to produce the polypeptide recombinantly. In a further aspect the present invention provides the use of nucleic acid encoding ES-2 polypeptide in production of ES-2.

Nucleic acids of the present invention include nucleic acids which include a sequence encoding a polypeptide which includes the amino acid sequence of SEQ ID NO. 2 and a polypeptide having at least 35% e.g. at least 70% sequence identity to SEQ ID NO. 2. Preferably the degree of sequence identity in either case is at least 50%, 60%, 70% or 80%, such as at least 90%, 95%, 98% or 99%.

Nucleic acids useful in the invention further include nucleic acids which include a sequence having at least 70% homology, more preferably at least 80%, such as at least 90%, 95%, 98% or 99% sequence homology to the nucleic acid sequences of SEQ ID NO. 1 or its complement.

Nucleic acid of the invention may encode the amino acid sequence of SEQ ID NO. 2, in which case it may include SEQ ID NO. 1 or a different nucleotide sequence, as permitted by degeneracy of the genetic code, or a polypeptide with ES-2 chemokine binding and/or inhibition which has an amino acid sequence which differs from SEQ ID NO. 2.

Where an aspect of the present invention is expressed in terms of nucleic acid with at least a specified % homology with SEQ ID NO. 1 or its complement, the actual sequence of SEQ ID NO. 1 or its complement may be excluded. In various embodiments the present invention provides non-naturally occurring nucleic acid encoding a ES-2 polypeptide, such as a polypeptide including the amino acid sequence of SEQ ID NO. 2 or an allelic variant thereof, or a non-naturally occurring polypeptide mutant, variant or derivative thereof.

One nucleic acid molecule provided by the present invention consists of the nucleotide sequence of SEQ ID NO. 1, or the RNA equivalent. Such a molecule may be provided as a component of a nucleic acid construct, for instance where the coding sequence is placed under regulatory control of a heterologous sequence, such as a promoter. A stop codon may immediately follow the coding sequence of SEQ ID NO. 1, e.g. TAA, as occurs naturally in the cloned sequence, or TAG or TGA, or additional coding sequence encoding a peptide tag, protein domain or other heterologous polypeptide sequence may follow, providing a nucleotide sequence encoding a fusion protein.

Nucleic acid sequences encoding all or part of a ES-2 gene can be readily prepared by the skilled person using the information and references contained herein and techniques known in the art (for example, see Sambrook, Fritsch and Maniatis, “Molecular Cloning, A Laboratory Manual”, Cold Spring Harbor Laboratory Press, 1989, and Ausubel et al, Short Protocols in Molecular Biology, John Wiley and Sons, 1992). These techniques include (i) the use of the polymerase chain reaction (PCR) to amplify samples of such nucleic acid, e.g. from genomic sources, (ii) chemical synthesis, or (iii) preparing cDNA sequences. Modifications to the wild type sequences described herein can be made, e.g. using site directed mutagenesis, to lead to the expression of modified polypeptides or to take account of codon preference in the host cells used to express the nucleic acid.

In general, short sequences for use as primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15-50 nucleotides) based on the sequence information provided herein to a region of the mRNA or genomic sequence encoding the mRNA which it is desired to clone, bringing the primers into contact with mRNA or cDNA obtained from schistosomes, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Such techniques may be used to obtain all or part of the sequences described herein.

Polynucleotides which are not 100% homologous to the sequences of the present invention but fall within the scope of the invention can be obtained in a number of ways.

Other schistosome variants (for example allelic forms) of the ES-2 gene described herein may be obtained for example by probing DNA libraries with probes including all or part of a nucleic acid of the invention under conditions of medium to high stringency (for example for hybridization on a solid support (filter) overnight incubation at 42° C. in a solution containing 50% formamide, 5×SSC (750 mM NaCl, 75 mM sodium citrate), 50 mM sodium phosphate (pH 7.6), 5×Denhardt's solution, 10% dextran sulphate and 20 μg/ml salmon sperm DNA, followed by washing in 0.03 M sodium chloride and 0.03 M sodium citrate (i.e. 0.2×SSC) at from about 50° C. to about 60° C.).

Thus the present invention may employ an isolated nucleic acid which hybridizes to the nucleotide sequence set forth in SEQ ID NO. 1 under the abovementioned hybridization and washing conditions. Such a nucleic acid is suitable for use as a probe for detecting an ES-2 gene, for example in Southern blots.

Alternatively, such polynucleotides may be obtained by site directed mutagenesis of the sequences of SEQ ID NO. 1 or allelic variants thereof. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides. Further changes may be desirable to represent particular coding changes which are required to provide, for example, conservative substitutions.

In the context of cloning, it may be necessary for one or more gene fragments to be ligated to generate a full-length coding sequence. Also, where a full-length encoding nucleic acid molecule has not been obtained, a smaller molecule representing part of the full molecule, may be used to obtain full-length clones. Inserts may be prepared from partial CDNA clones and used to screen cDNA libraries. The full-length clones isolated may be subcloned into expression vectors and activity assayed by transfection into suitable host cells, e.g. with a reporter plasmid.

Preferably, a polynucleotide of the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A control sequence “operably linked” to a coding sequence is ligated in such a way that expression of the coding sequence is achieved under condition compatible with the control sequences.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. 'phage, phagemid or baculoviral, cosmids, YACS, BACs, or PACs as appropriate.

The vectors may be provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo, for example in methods of gene therapy. Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others.

For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Baculovirus expression is described for example in Alcami et al. 1998, J. Immunol. 160:624-633.

Vectors may be transformed into a suitable host cell as described above to provide for expression of a polypeptide of the invention. Thus, in a further aspect the invention provides a process for preparing polypeptides according to the invention which includes cultivating a host cell transformed or transfected with an expression vector as described above under conditions to provide for expression by the vector of a coding sequence encoding the polypeptides, and recovering the expressed polypeptides. Polypeptides may also be expressed in in vitro systems, such as reticulocyte lysate.

Following production of a polypeptide of the invention it may be tested for ES-2 activity, e.g. by determination of ability to bind a chemokine such as a CC chemokine such as MIP-1α and/or RANTES, and/or a CXC chemokine such as IL-8, or inhibit chemokine activity in an assay, such as in an assay substantially as described in the experimental section herein. The polypeptide may be tested for ability to inhibit IL-8 mediated neutrophil migration and/or infiltration, e.g. into an air pouch in vivo in an animal, especially a non-human animal such as a mouse. Additionally, or alternatively, the polypeptide produced may be tested for immunological characteristics of ES-2 polypeptide, e.g. ability to bind one or more antibody molecules that recognise ES-2 polypeptide.

In various further aspects the present invention relates to screening and assay methods and means, and substances identified thereby, especially substances that affect ability of ES-2 polypeptide to bind and/or inhibit chemokine activity.

Thus, further aspects of the present invention provide the use of ES-2 polypeptide (e.g. a fragment of a polypeptide of the invention as disclosed that binds a chemokine, and/or encoding nucleic acid therefor), in screening or searching for and/or obtaining/identifying a substance, e.g. peptide or chemical compound, which interacts and/or binds with the polypeptide or peptide and/or interferes with its function or activity or that of another substance, e.g. polypeptide or peptide, which interacts and/or binds with the polypeptide or peptide of the invention. For instance, a method according to one aspect of the invention includes providing a polypeptide of the invention and bringing it into contact with a substance, which contact may result in binding between the polypeptide or peptide and the substance. Binding may be determined by any of a number of techniques available in the art, both qualitative and quantitative.

In various aspects the present invention is concerned with provision of assays for substances which interact with or bind a polypeptide of the invention and/or modulate one or more of its activities.

One aspect of the present invention provides an assay which includes:

(a) bringing into contact a polypeptide or peptide according to the invention and a putative binding molecule or other test substance; and

(b) determining interaction or binding between the polypeptide or peptide and the test substance.

A substance which interacts with the polypeptide or peptide of the invention may be isolated and/or purified, manufactured and/or used to modulate its activity as discussed.

It is not necessary to use the entire proteins for assays of the invention which test for binding between two molecules as above or test for ES-2 polypeptide activity (see below). Fragments may be generated and used in any suitable way known to those of skill in the art. Suitable ways of generating fragments include, but are not limited to, recombinant expression of a fragment from encoding DNA. Such fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments (e.g. up to about 20 or 30 amino acids) may also be generated using peptide synthesis methods which are well known in the art.

The precise format of the assay of the invention may be varied by those of skill in the art using routine skill and knowledge. For example, the interaction between the polypeptides may be studied in vitro by labelling one with a detectable label and bringing it into contact with the other which has been immobilised on a solid support. Suitable detectable labels include ³⁵S-methionine which may be incorporated into recombinantly produced peptides and polypeptides. Recombinantly produced peptides and polypeptides may also be expressed as a fusion protein containing an epitope which can be labelled with an antibody.

The protein which is immobilized on a solid support may be immobilized using an antibody against that protein bound to a solid support or via other technologies which are known per se. A preferred in vitro interaction may utilise a fusion protein including glutathione-S-transferase (GST). This may be immobilized on glutathione agarose beads. In an in vitro assay format of the type described above a test compound can be assayed by determining its ability to diminish the amount of labelled peptide or polypeptide which binds to the immobilized GST-fusion polypeptide. This may be determined by fractionating the glutathione-agarose beads by SDS-polyacrylamide gel electrophoresis. Alternatively, the beads may be rinsed to remove unbound protein and the amount of protein which has bound can be determined by counting the amount of label present in, for example, a suitable scintillation counter.

Determination of the ability of a test compound to interact and/or bind with an ES-2 polypeptide or fragment may be used to identify that test compound as a candidate for a modulator of ability of ES-2 polypeptide to bind and/or inhibit a chemokine, e.g. a CC chemokine such as MIP-1α and/or RANTES, and/or a CXC chemokine such as IL-8. Generally, then identification of ability of a test compound to bind a polypeptide or fragment of the invention is followed by one or more further assay steps involving determination of whether or not the test compound is able to inhibit ES-2 binding to a chemokine and/or affect ES-2 activity (such activity being ability to bind and/or inhibit activity of a chemokine, e.g. a CC chemokine such as MIP-1α and/or RANTES, and/or a CXC chemokine such as IL-8). Naturally, assays involving determination of ability of a test substance to modulate ES-2 activity may be performed where there is no knowledge about whether the test substance can bind or interact with ES-2 polypeptide, but a prior binding/interaction assay may be used as a “coarse” screen to test a large number of substances, reducing the number of candidates to a more manageable level for a functional assay involving determination of ability to modulate ES-2 polypeptide activity. An assay according to the present invention may also take the form of an in vivo assay. The in vivo assay may be performed in a cell line in which the relevant polypeptides or peptides are expressed from one or more vectors introduced into the cell. A preferred assay of the invention includes determining the ability of a test compound to modulate ES-2 polypeptide activity of an isolated or purified polypeptide of the invention (which may be a full-length ES-2 or an active portion thereof).

Another assay method of screening in accordance with the present invention comprises:

(a) bringing into contact a substance comprising an ES-2 polypeptide, a second substance comprising a chemokine polypeptide which is able to bind the ES-2 polypeptide; and a test compound, under conditions in which in the absence of the test compound being an inhibitor, the two said substances interact;

(b) determining interaction between said substance.

A quantitative assay, that is an assay in which the degree of binding can be measured, whether increased or decrease, allows for identification of test compounds that are able to potentiate or inhibit ES-2 polypeptide binding to chemokine polypeptide.

As noted already, it is not necessary to use full-length ES-2 and chemokine—fragments of each that bind or interact are sufficient.

A method of screening for a substance which modulates activity of a polypeptide may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

In a further aspect of the invention there is provided an assay method which includes:

(a) incubating an isolated polypeptide which has ES-2 polypeptide activity and a test compound in the presence of a chemokine such as a CC chemokine such as MIP-1α and/or RANTES, and/or a CXC chemokine such as IL-8, under conditions in which chemokine activity is inhibited by the ES-2 polypeptide; and

(b) determining chemokine activity.

An inhibitor or potentiator of ES-2 polypeptide activity may be identified (or a candidate substance suspected of being a ES-2 polypeptide inhibitor or potentiator may be confirmed as such) by determination of chemokine activity compared with a control experiment in which the test compound is not applied.

Combinatorial library technology (Schultz, J S (1996) Biotechnol. Prog. 12:729-743) provides an efficient way of testing a potentially vast number of different substances for ability to modulate activity of a polypeptide. The amount of test substance or compound which may be added to an assay of the invention will normally be determined by trial and error depending upon the type of compound used. Compounds which may be used may be natural or synthetic chemical compounds used in drug screening programmes. Extracts of plants which contain several characterised or uncharacterised components may also be used. Other candidate inhibitor compounds may be based on modelling the 3-dimensional structure of a polypeptide or peptide fragment and using rational drug design to provide potential inhibitor compounds with particular molecular shape, size and charge characteristics.

Following identification of a substance which modulates or affects polypeptide activity, the substance may be investigated further. Furthermore, it may be manufactured and/or used in preparation, i.e. manufacture or formulation, of a composition such as a medicament, pharmaceutical composition or drug. These may be administered to individuals, as already discussed.

A substance identified using as a modulator of ES-2 polypeptide activity may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use. The designing of mimetics to a known pharmaceutically active compound is a known approach to the development of pharmaceuticals based on a “lead” compound. This might be desirable where the active compound is difficult or expensive to synthesize or where it is unsuitable for a particular method of administration. Mimetic design, synthesis and testing may be used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

Mimetics of substances identified as having ability to modulate polypeptide activity using a screening method as disclosed herein are included within the scope of the present invention. A polypeptide, peptide or substance able to modulate activity of a polypeptide according to the present invention may be provided in a kit, e.g. sealed in a suitable container which protects its contents from the external environment. Such a kit may include instructions for use.

Further aspects and embodiments of the present invention will be apparent to those skilled in the art. The following experiments provide support for and exemplification by way of illustration of aspects and embodiments of the invention.

All documents mentioned in this specification are incorporated by reference.

### EXPERIMENTAL

Identification of ES-2 as a Chemokine-Binding Molecule

Schistosoma mansoni eggs were cultured in vitro and the secretions from the eggs (Egg Secretions; ES) were collected. ES and purified ES-2 were incubated with ¹²⁵I-MIP-1α or ¹²⁵I-IL-8 and cross-linked with EGS. Following resolution by SDS-PAGE the gel was developed by autoradiography. The unbound ¹²⁵I-chemokine was detected at approximately 8 kDa at the bottom of the gels. In whole ES and purified ES-2 bound ¹²⁵I-chemokine 2 bands were detected at approximately 24 and 44 kDa. This activity was unique to ES as no bound chemokine was detected in the binding media used, only background non-specific activity (e.g. band at approx. 100 kDa). The addition of cold MIP-1α or IL-8 to ES-2 blocked the subsequent formation of a ¹²⁵I-chemokine-ES-2 complex.

As MIP-1α and IL-8 are approximately 8 kDa, the predicted size of the chemokine-binding molecules minus bound chemokine would be approximately 18 and 36 kDa. This size corresponded with a molecule, designated ES-2, previously shown to be secreted from schistosome eggs. Based on molecular weight, in native and reduced SDS-PAGE, ES-2 is a previously described egg glycoprotein that is present in the eggs of the three major of species of schistosome that infects humans (6, 7).

ES-2 was isolated from schistosome egg antigens using cation-exchange chromatography. The inventors then showed in chemokine-cross-linking assays that purified ES-2 bound ¹²⁵I-MIP-1α and ¹²⁵I-IL-8. The addition of unlabelled (cold) IL-8 or MIP-1α to the cross-linking assay ablated binding of radio-labeled chemokine by ES-2, demonstrating ES-2 was specifically binding to chemokines.

ES-2 was cross-linked to ¹²⁵I-MIP-1α and the complex resolved by SDS-PAGE. The gel was cut and used as follows:

i. Developed as an autoradiograph to show bound ¹²⁵I-chemokine at 24 and 44 kDA;

ii. Silver-stained to confirm the size of the ES-2-chemokine complex;

iii. Electro-transferred to nitrocelluose paper (Western Blotting) and probed with sera from mice immunized with ES-2.

These data confirmed that ES-2 is a chemokine-binding glycoprotein.

ES-2 Does Not Bind to the Chemokine GAG-Binding Domain.

Chemokines can interact with glucosaminoglycans (GAGs) such as heparin or heparan sulfate through their carboxy terminus. This interaction is thought to facilitate chemokine localization to endothelial cells and does not interfere with chemokines binding to their receptors.

¹²⁵I-MIP-1α was pre-incubated with 1 mg/ml heparin or heparan sulfate (Sigma) before incubation with ES-2. Following cross-linking the samples were resolved by SDS-PAGE and the autoradiograph developed. The heparin or heparan sulfate did not interfere with ES-2 binding to MIP-1α; ES-2 binding to chemokine is GAG-independent. No ¹²⁵I-MIP-1α-ES-2 complex formed when cold MIP-1α was added.

ES-2 Can Block the Binding of Chemokines to Receptors on Cells.

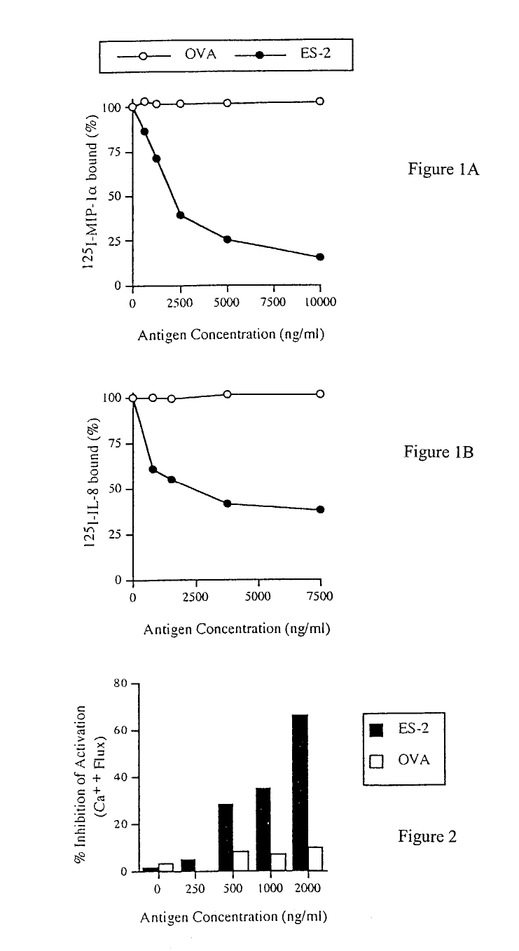

A competition chemokine-binding assay with U937 cells (human monocytic cell line) was used. ES-2 inhibited binding of ¹²⁵I-MIP-1α or ¹²⁵I-IL-8 to the cells in a dose dependent manner (FIG. 1). Ovalbumin (OVA), a control glycoprotein of comparable molecule weight, did not affect chemokine binding (FIG. 1).

ES-2 Can Inhibit Chemokine-Mediated Activation and Migration of Cells.

In vitro studies were used to determine if ES-2 could impair functional chemokine activity.

To address if ES-2 blocked chemokine-mediated activation of cells human peripheral mononuclear cells (PBMCs) were labeled with Fluro 4AM. These cells were stimulated by the chemokine RANTES and cellular activation (Ca⁺⁺ flux) was detected by Flow cytometry. The addition of ES-2 to the RANTES inhibited its ability to activate PBMCs in a dose-dependent manner, whereas OVA did not alter chemokine activity (FIG. 2).

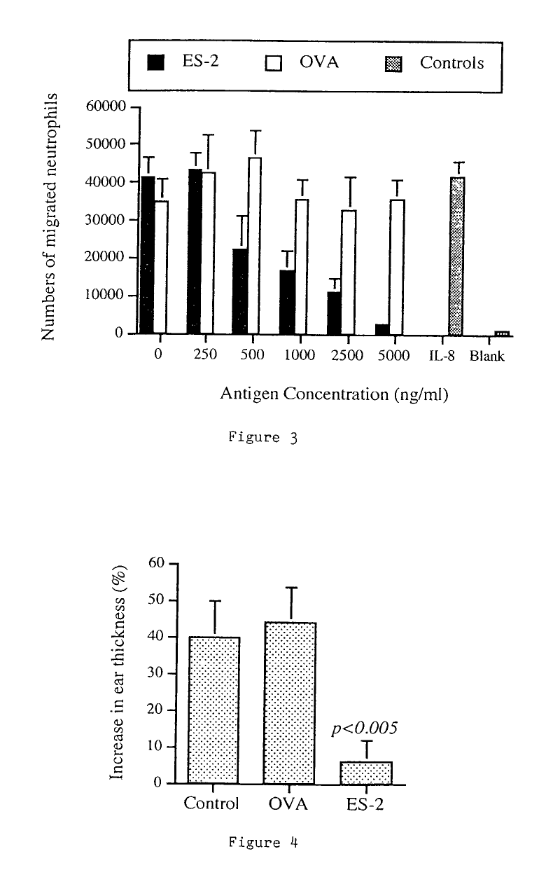

Neutrophils were isolated from PBMCs and stimulated to migrate through the membranes of modified Boyden chambers by IL-8. The addition of ES-2 to IL-8 inhibited chemokine-stimulated cell migration in a dose dependent fashion (FIG. 3). Similar to previous studies, the control glycoprotein OVA did not inhibit chemokine activity.

ES-2 Inhibits Inflammation in Vivo.

A murine contact hypersensitivity (CHS) model was used to test ES-2 modulation in an in vivo inflammatory response.

Mice that were pre-sensitized to 2,4-dinitroflurobenzene (DNFB) were intravenously injected with 15 μg of ES-2 or OVA (in 0.2 mls sterile PBS) 20 minutes prior elicitation of CHS by application of a DNFB suspension to the right ear. In the control PBS- or OVA-injected mice there was 40-45% increased in ear thickness by 24 hours after elicitation of CSH (FIG. 4). The administration of ES-2 to mice significantly (p<0.005; Students t-test) reduced the inflammation relative to the control groups.

Isolation of the Gene Encoding ES-2.

The 2 bands that constitute ES-2 on a SDS-PAGE gel were excised and used for mass spectrometry to obtain peptide sequence. The resulting peptide data was used to screen databases revealing no homology to known proteins.

However, one ES-2 peptide (ITGLGHGTCIDDFTK) (SEQ ID NO: 3) matched an EST clone deposited in the Schistosoma EST databases. The clone was obtained and sequenced. The nucleotide sequence (SEQ ID NO: 1) and deduced amino acid sequence (SEQ ID NO: 2) of ES-2 were determined.

Confirmation that the sequenced gene encoded ES-2 was shown by the presence in the deduced amino acid sequence of three peptides that were initially identified by mass spectrometry of ES-2.

Expression of Recombinant ES-2 Protein in Baculovirus

ES-2 protein cDNA was expressed in a baculovirus system (Alcami et al. 1998, J. Immunol. 160:624-633).

The ES-2 ORF was PCR-amplified with specific oligonucleotides and cloned into a baculovirus expression vector under the control of the strong polyhedrin promoter and fused to a C-terminal 6xhistidine tag (pES2his). An additional construct was also prepared in which, in addition to the C-terminal 6xhistidine tag, the signal peptide of CD33 was cloned in frame at the N-terminus of the ES-2 ORF to provide a signal for secretion (pCD33ES2his). Recombinant baculoviruses (AcCD33ES2his and AcES2his) were generated following standard procedures.

The recombinant ES-2 protein was secreted from Spodoptera frugiperda insect cells infected with both AcCD33ES2his and AcES2his. Recombinant protein was purified by affinity chromatography in nickel chelate columns from supernatants of baculovirus-infected insect cell cultures following standard procedures.

Purified recombinant ES-2 protein was visualized by Coomassie blue staining or by Western blotting with antiserum against natural ES-2 or 6xhistidine tag. The purified recombinant ES-2 protein had a size similar to that of the native protein (approximately 25 kDa) and was recognized by a rabbit antiserum specific against ES-2 and antibodies specific for the 6xhistidine tag.

Moreover, purified recombinant ES-2 bound chemokines as demonstrated by cross-linking to ¹²⁵I iodinated interleukin 8 followed by SDS-PAGE analysis and autoradiography.

Detection of Antibodies against ES-2 in Mice with Schistosome Infection

The purified recombinant ES-2 protein was found to be recognized by sera from mice infected with Schistosoma mansoni in Western blots. This provides further indication of a role for ES-2 in therapy and diagnosis of schistosome infection in humans.

Recombinant ES-2 Inhibits Neutrophil Recruitment In Vivo

Using a mouse air pouch model systemic (intravenous) administration of recombinant ES-2 (ES-2 His) was shown to significantly reduce chemokine (IL-8) stimulated infiltration of neutrophils (P<0.001, ANOVA). In contrast, in mice injected with a control protein, ovalbumin (OVA), IL-8 treatment induced marked neutrophil infiltration of the air pouch. rES-2 treatment reduced IL-8 mediated neutrophil recruitment to levels non-significantly higher than neutrophil infiltration induced by injection of PBS into the air pouch.

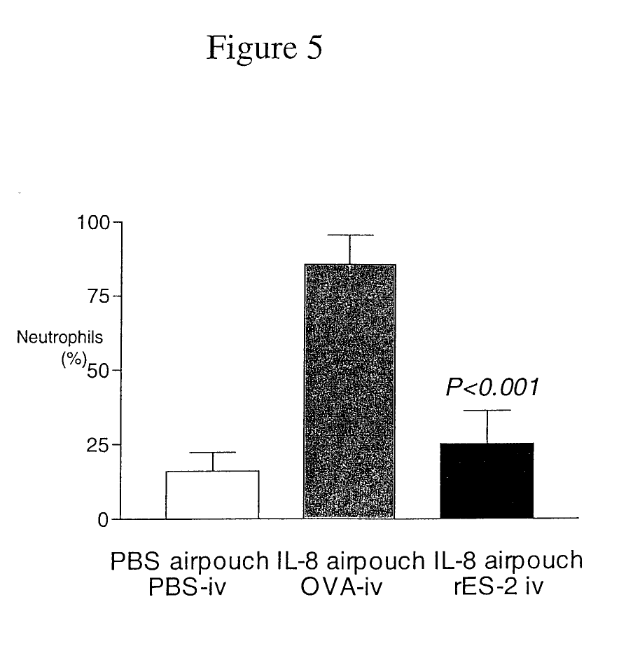

FIG. 5 shows that neutrophil infiltration was significantly reduced (P<0.001) in rES-2 treated mice compared to control OVA-treated mice (ANOVA). Data are mean+SD from 6 mice per group.

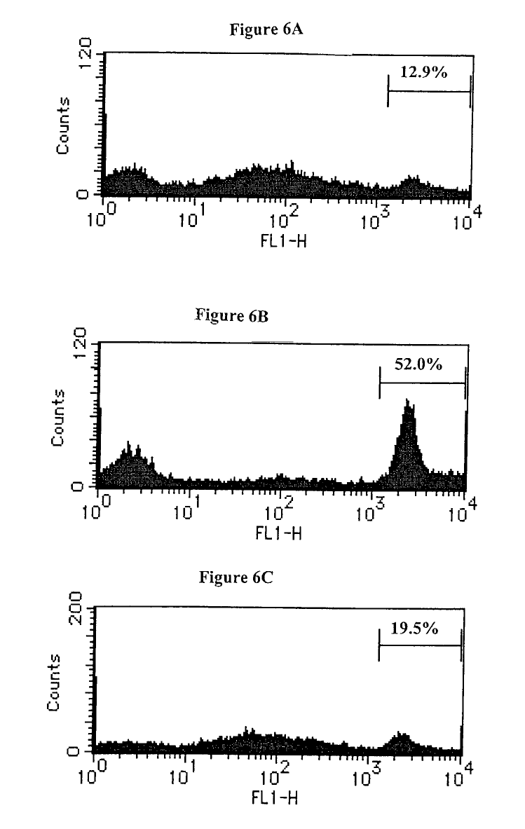

FIG. 6 shows results of flow cytometry detection of GR-1 positive neutrophils (expressed as percentage) in the air pouch lavage of mice. PBS or IL-8 was injected into the air pouch. Mice were injected intravenously with 200 μg of PBS containing 20 μg of rES-2 or Ovalbumin.

### MATERIALS AND METHODS

Parasite Eggs and Preparation of ES.

Schistosome eggs were obtained as described in reference (8). Eggs were counted and the stage of the development of the eggs determined using Vogel's scheme. Eggs were used immediately for in vitro egg cultures or used for preparation of whole egg antigens (soluble egg antigens [SEA]). To prepare SEA eggs were washed in PBS and the eggshell was disrupted with a combination of the use of a percussion mortar and sonication. The sonicated solution was repeatedly centrifuged (3000 g). SEA was stored at −20° C.

For in vitro cultures of eggs all reagents were sterile and procedures were performed under aseptic conditions. Isolated eggs were washed at least three times in minimum essential medium (MEM); supplemented with Penicillin-Streptomycin (50U/ml-50 mg/ml), Gentamycin (50 μl/ml) and Glutamine (2 mM). Eggs were counted and cultured at 37° C. in dialysis tubing. The media in the flasks was MEM, as above, but supplemented with 10% foetal calf serum (FCS; Sigma). After 2 days the supernatant was collected, dialysed and concentrated using ultra-filtration membranes (Amicon, Gloucs.,UK). This preparation was termed egg secretions (ES) and was stored at −20° C.

Isolation of ES-2.

Egg antigens were fractionated by cation-exchange chromatography using a NaCL gradient. A fraction was identified with the 2 bands comprising ES-2 on reduced or native SDS-PAGE. Anti-ES-2 mouse sera was prepared by immunization of Balb/c mice with purified ES-2. The 2 bands composing ES-2 in SDS-PAGE were excised and subjected to Mass Spectrometry analysis using a ThermoFinnigan LCQ Classic with Protana nanospray interface. Three peptides identified were used. An EST clone, with sequence homology with 1 ES-2 peptide, was sequenced and the amino acid sequence predicted.

Chemokine Binding Assays:

Cross-Linking Experiments

Cross-linking binding assays were performed as described (9, 10). In brief, ES or ES-2 were incubated with iodinated-chemokines in binding medium (RPMI 1640 containing 20 mM Hepes (pH 7.4) and 0.1% BSA). Cold IL-8 or MIP-1α was added as controls. Cross-linking to ¹²⁵I-IL-8 or ¹²⁵I-MIP-1α. (NEN, Boston, Mass.) was performed with 1-ethyl-3-(3-dimethylaminopropyl)-carbodiimide (EDC) or ethylene glycol-bis succinamidyl succinate (EGS) (1 mg/ml) in 25 μl. Samples were analyzed by 12% acrylamide SDS-PAGE and developed by autoradiography. Ovalbumin (OVA, Sigma) was included in chemokine studies as a control glycoprotein of comparable molecule weight as ES-2. To address if ES-2 bound GAGs, heparin or Heparan sulfate (Sigma; Concentration 1 mg/ml) were incubated with ES-2 and ¹²⁵I-MIP-1α

U937 cells were used in the competition assays. OVA or ES-2 were pre-incubated with 100 pM ¹²⁵I-chemokine in 100 μl for 1 h at 4° C. Subsequently, 2.5×10⁶ U937 cells were added in 50 μl and incubated for 2 h at 4° C. Bound ¹²⁵I-chemokine was determined by phthalate oil centrifugation (11).

Purification of Human PBMCs and Isolation of Neutrophils.

Peripheral blood mononuclear cells (PBMCs) were isolated from whole blood using Lymphoprep according to the manufacturer's instructions. PBMCs were washed in Hanks buffered saline solution (HBSS, Sigma). Neutrophils were obtained from donors for use in calcium flux assay.

Calcium Flux Assay

Ca⁺⁺ flux by chemokine-activated cells was determined using previous described methods (12). Isolated PBMCs cells were washed in HBSS containing (1% FCS). Cells were suspended (1×10⁷ cells/ml) in calcium and magnesium free HBSS (Gibco, UK). Cells were labeled by incubation (20 minutes, 37° C.) with Fluro-4AM (Molecular Probes; 4 μmolar final concentration) and Pluronic F-127 (Molecular Probes; 0.02% final concentration). Cells were diluted (1 in 5) in HBSS containing 1% FCS and incubated for 40 minutes at 37° C. Cells were washed and re-suspended in HEPES buffered saline (1.1×10⁶ cells/ml) and incubated for 10 minutes at 37° C. The labeled cell suspension was used in Ca⁺⁺ flux assays.

Labeled PBMCs were activated by the addition of RANTES-alone (PeproTech; 100 ng/ml final concentration) or RANTES preincubated with ES-2 or OVA as a control antigen (both antigens 250-2000 ng/ml) for 6 minutes at room temperature. 50 l of chemokines±antigens was added to 95 μl of stock PBMCs (1.1×10⁶ cells/ml). Ca⁺⁺ flux by activated cells was analysed using a FACScan and Cell Quest software (Becton Dickinson). Data is presented as the % inhibition of RANTES elicited Ca⁺⁺ flux by the addition of ES-2 or OVA.

Neutrophil Migration Assay

Neutrophils were isolated from human PBMCs. IL-8 (PeproTech, stock concentration 100 ng/ml) was mixed with a range of concentrations of ES-2 or OVA (250-5000 ng/ml) for 15 minutes at 37° C. At the end of the incubation period 29 μl of solution was added to the bottom well of the transwell migration chamber (ChemoTX, Nuero Probe Inc, Md., USA; with 3 mm pore size). Triplicate wells were used per dilution and negative control (no IL-8; blank) and positive controls (IL-8-alone) were included. To each well 5×10⁴ neutrophils were added. Cell migration occurred over 1 hour (37° C.; 5% CO₂). The membrane was removed and treated with EDTA and cell were collected by centrifugation. Cell migration was determined by cell counts using Fast-Read counting chabers (ISL, Paignon, UK). Data are presented as mean and SD from triplicate wells.

Contact Hypersensitivity.

The in vivo model of contact hypersensitivity (CHS) was essentially as described in references (13, 14). 6-8 week-old female Balb/c mice were used (Harlan). Mice were sensitized by topical application of 25 μl of 0.5% 2,4-dintrofluorobenzenes (DNFB; Sigma) to the shaved abdomen. DNFB solutions were prepared in acetone/olive oil (4:1). CHS was elicited 5 days later by application of 20 μl of 0.02% DNFB solution to the right ear and 20 μl of vehicle (acetone/olive oil [4:1]) to the left ear. Ear swelling was measured with a dial thickness gauge (Mitutoyo, Kawasaki, Japan) before and 24 hours after elicitation of CHS. Ear swelling was measured by determining the increase in left (vehicle) and right (DNFB) post-challenge relative to pre-challenge thickness. The post-challenge increase in ear thickness of the right ear relative to the left was expressed as a percentage.

To address if ES-2 altered in vivo CHS responses sensitized mice were i.v. injected with 15 μg of ES-2 or OVA (in 0.2 mls sterile PBS) 20 minutes before the application of DNFB to the ear. ES-2 was determined to be endotoxin-free by commercial kit (COATEST-Endotoxin; Chromogenic AB, Molndal, Sweden). Data are from 6-7 mice per group and are presented as mean and SE. Student's t-test was used to determine statistical differences between groups.

Air Pouch Model

Dorsal air pouches were induced using methods described [15, 16]. 4 mls of sterile-filtered air was injected subcutaneously into the back of female C57BL/6 mice. 3 Days later the air pouch was re-inflated with 3 mls of sterile air. 3 days later, groups of 6 mice were intravenously injected with 200 μl of endotoxin-free phosphate-buffered saline (PBS;Sigma) or 200 μl of PBS containing 20 μg of recombinant ES-2 His or 20 μg of ovalbumin (all endotoxin free). 30 minutes later 1 ml of PBS, or 1 ml of PBS with 1 μg recombinant human IL-8 (Peprotech) was injected into the air pouch. 3 hours later mice were killed and air pouches were lavaged with PBS. The aspirate was centrifuged and cells recovered and counted. Cells were cytospun onto slides and stained with Wright-Giemsa. The number of infiltrating neutrophils were counted and are expressed as a percentage. In addition, aspirated cells were surface stained for the granulocyte surface marker GR1 using FITC-conjugated RB6-8C5 mAb (PharMingen). Granulocytes were gated and the number of GR-1 positive cells determined by flow cytometry.

### REFERENCES

1. Alcami and Koszinowski. 2000. Immunol Today 21:447.

2. Fallon. 2000. Immunology Today. 21:29.

3. Qiu et al. 2001. Am J Pathol 158:1503.

4. Proudfoot et al. 2000. Immunological Reviews 177:246.

5. Gerard and Rollins. 2001. Nature Immunology 2:108.

6. Dunne et al. 1991. Parasitology 103 Pt 2:225.

7. Hamilton et al. 1999. Parasitology 118:83.

8. Fallon et al. Eur J Immunol 28:1408.

9. Alcami and Smith. 1995. J Virol 69:4633.

10. Upton et al. 1992. Science 258:1369.

11. Patel et al. 1990. J Gen Virol 71:2013.

12. vandenberghe and Ceuppens. 1990. J Immunol Methods 127:197.

13. Garrigue et al. 1994. Contact Dermatitis 30:231.

14. Varona et al. 2001. J Clin Invest 107:R37.

15. Edwards et al. 1981. J. Pathol. 134:147-156.

16. Nakamura et al. 2001. Proc. Natl. Acad. Sci. USA. 18: 15143-15148.

| | | |
|---|---|---|
| Met Ser Ala Asn Ser Met Phe Leu Ile Ala Val Leu Ser Tyr Thr Leu Ile | 17 | SEQ ID NO:1 |
| ATG TCA GCT AAT TGG ATG TTT CTT ATT GCC GTA TTG TCA TAC ACA TTG ATA | | SEQ ID NO:2 |
| Ser Gln Leu Gly Ile Thr Thr Ser Asp Ser Cys Lys Tyr Cys Leu Gln Leu | 34 | |
| AGT CAA TTG GGG ATA ACT ACA TCG GAT TCA TGC AAA TAT TGT CTA CAA TTG | | |
| Tyr Asp Glu Thr Tyr Glu Arg Gly Ser Tyr Ile Glu Val Tyr Lys Ser Val | 51 | |
| TAC GAT GAA ACG TAT GAG AGG GGT TCA TAT ATT GAA GTC TAC AAA AGC GTT | | |
| Gly Ser Leu Ser Pro Pro Trp Thr Pro Gly Ser Val Cys Val Pro Phe Val | 68 | |
| GGC TCA CTC TCA CCA CCA TGG ACA CCT GGA TCT GTT TGT GTA CCC TTC GTA | | |
| Asn Asp Thr Lys Arg Glu Arg Pro Tyr Trp Tyr Leu Phe Asp Asn Val Asn | 85 | |
| AAT GAC ACG AAG AGA GAG CGT CCA TAC TGG TAT TTA TTT GAC AAC GTC AAT | | |
| Tyr Thr Gly Arg Ile Thr Gly Leu Gly His Gly Thr Cys Ile Asp Asp Phe | 102 | |
| TAC ACA GGT CGG ATT ACT GGT CTC GGA CAT GGT ACC TGC ATT GAT GAC TTC | | |
| Thr Lys Ser Gly Phe Lys Gly Ile Ser Ser Ile Lys Arg Cys Ile Gln Thr | 119 | |
| ACG AAA TCC GGA TTC AAA GGC ATT TCC TCT ATT AAA CGG TGT ATT CAA ACA | | |
| Lys Asp Gly Lys Val Glu Cys Ile Asn Gln Pro Lys Leu Arg Arg Thr Tyr | 136 | |
| AAG GAT GGA AAA GTT GAA TGT ATC AAT CAA CCG AAA CTG AGA AGG ACA TAC | | |
| Cys Arg Phe | 139 | |
| TGT CGA TTC | | |

1. A method of producing a polypeptide which has chemokine-binding activity, the method comprising:
(a) causing expression from nucleic acid which encodes a polypeptide which is a chemokine-binding molecule in a suitable expression system to produce the polypeptide,
wherein the polypeptide is a chemokine-binding molecule which comprises the amino acid sequence of SEQ ID NO. 2 or consists of a portion of the amino acid sequence of SEQ ID NO. 2 that is chemokine-binding, or which has an amino acid sequence which differs from the amino acid sequence of SEQ ID NO. 2 but has at least 35% and preferably at least 70% identity with the amino acid sequence of SEQ ID NO. 2 and is chemokine-binding; and
(b) testing the polypeptide for chemokine-binding activity or ability to inhibit chemokine activity.

2. A method according to claim 1 wherein the polypeptide has at least 90% identity with the amino acid sequence of SEQ ID NO. 2.

3. A method according to claim 1 wherein said polypeptide comprises the amino acid sequence of SEQ ID NO. 2.

4. A method according to claim 3 wherein said nucleic acid comprises the nucleotide sequence of SEQ ID NO. 1.

5. A method according to claim 1 comprising isolating the polypeptide.

6. A method according to claim 5 comprising testing the polypeptide for ability to inhibit chemokine activity.

7. A method according to claim 1 wherein the polypeptide inhibits chemokine activity.

8. A method according to claim 1 further comprising formulating the polypeptide into a composition comprising at least one additional component.

10. A method according to claim 1 further comprising bringing the polypeptide into contact with a chemokine to inhibit activity of the chemokine.

11. A method according to claim 10 wherein the chemokine is contacted with the polypeptide in vitro.

12. A method according to claim 10 comprising administering the polypeptide to an individual.

13. A method according to claim 12 wherein the individual has a disease or disorder involving activity of a chemokine, which activity is inhibited by the polypeptide.

14. A method according to claim 1 further comprising employing the polypeptide in a diagnostic product.

15. A nucleic acid construct suitable for use in a method of producing a polypeptide as defined in claim 1, the construct comprising a nucleotide sequence which encodes the polypeptide and which is operably linked to regulatory sequences for expression of the encoded polypeptide.

16. A nucleic acid construct according to claim 15 wherein the encoded polypeptide comprises the amino acid sequence of SEQ ID NO. 2.

17. A nucleic acid construct according to claim 16 wherein said nucleic acid comprises the nucleotide sequence of SEQ ID NO. 1.

18. A nucleic acid construct according to claim 15 wherein the polypeptide which is a chemokine-binding molecule consists of a portion of the amino acid sequence of SEQ ID NO. 2 that is chemokine-binding.

19. A nucleic acid construct according to claim 15 wherein the polypeptide which is a chemokine-binding molecule has an amino acid sequence which differs from the amino acid sequence of SEQ ID NO. 2 but has at least 35% and preferably at least 70% identity with the amino acid sequence of SEQ ID NO. 2.

20. A nucleic acid construct according to claim 19 wherein the polypeptide has at least 90% identity with the amino acid sequence of SEQ ID NO. 2.

21. A host cell transformed with a nucleic acid construct according to claim 15.

22. Use of a nucleic acid construct according to claim 15 in a method for producing a polypeptide which is a chemokine-binding molecule.

23. A isolated polypeptide produced by a method according to claim 1.

24. A method of treatment of a disease or disorder comprising administration of a polypeptide according to claim 23 to an individual, wherein the individual has a disease or disorder involving activity of a chemokine, which activity is inhibited by the polypeptide.

25. Use of a polypeptide according to claim 23 in the manufacture of a medicament for treatment of a disease or disorder involving activity of a chemokine, which activity is inhibited by the polypeptide.

26. Use of a polypeptide according to claim 23 in screening for or obtaining a substance which binds the polypeptide and optionally affects chemokine-binding ability of the polypeptide.

27. An assay method for a substance which binds a polypeptide, which polypeptide is a chemokine-binding molecule which comprises the amino acid sequence of SEQ ID NO. 2 or consists of a portion of the amino acid sequence of SEQ ID NO. 2 that is chemokine-binding, or which has an amino acid sequence which differs from the amino acid sequence of SEQ ID NO. 2 but has at least 35% and preferably at least 70% identity with the amino acid sequence of SEQ ID NO. 2 and is chemokine-binding, the method comprising:
(a) bringing into contact the polypeptide and a putative binding molecule or other test substance; and
(b) determining binding between the polypeptide and the test substance.

28. An assay method for a substance that affects chemokine-binding of a polypeptide, wherein the polypeptide is a chemokine-binding molecule which comprises the amino acid sequence of SEQ ID NO. 2 or consists of a portion of the amino acid sequence of SEQ ID NO. 2 that is chemokine-binding, or which has an amino acid sequence which differs from the amino acid sequence of SEQ ID NO. 2 but has at least 35% and preferably at least 70% identity with the amino acid sequence of SEQ ID NO. 2 and is chemokine-binding, the method comprising:
(a) bringing into contact a substance comprising the polypeptide, a second substance comprising a chemokine polypeptide which is able to bind the polypeptide which is a chemokine-binding molecule; and a test compound, under conditions in which in the absence of the test compound being an inhibitor, the two said substances interact;
(b) determining interaction between said substance.

29. An assay method for a substance that affects ability of a polypeptide to inhibit chemokine activity, wherein the polypeptide is a chemokine-binding molecule which comprises the amino acid sequence of SEQ ID NO. 2 or consists of a portion of the amino acid sequence of SEQ ID NO. 2 that is chemokine-binding, or which has an amino acid sequence which differs from the amino acid sequence of SEQ ID NO. 2 but has at least 35% and preferably at least 70% identity with the amino acid sequence of SEQ ID NO. 2 and is chemokine-binding, the method comprising:
(a) incubating the polypeptide and a test compound in the presence of a chemokine such as a CC chemokine such as MIP-1α and/or RANTES, and/or a CXC chemokine such as IL-8, under conditions in which chemokine activity is inhibited by the polypeptide; and
(b) determining chemokine activity.
